(19) United States
(12) Patent Application Publication
(10) Pub. No.: US 2008/0152615 A1
(43) Pub. Date: Jun. 26, 2008

(54) Composition for Adjuvant Containing Poly-Gamma-Glutamic Acid

(75) Inventors: Moon-Hee Sung, Daejeon (KR); Chul-Joong Kim, Daejeon (KR); Ha Ryoung Poo, Daejeon (KR); Seung Pyo Hong, Daejeon (KR); Jong Soo Lee, Gyeonggi-do (KR); Ji Youn Kim, Gyeonggi-do (KR)

Correspondence Address: INTELLECTUAL PROPERTY / TECHNOLOGY LAW, PO BOX 14329, RESEARCH TRIANGLE PARK, NC, 27709, US

(73) Assignees: BIOLEADERS CORPORATION, Daejeon (KR); M.D. LAB CO., LTD, Daejeon (RE); Korea Research Institute Of Bioscience and Biotechnology, Yuseong-gu, Daejeon (KR)

(21) Appl. No.: 11/817,021

(22) PCT Filed: Dec. 6, 2005
(86) PCT No.: PCT/KR2005/004160

§ 371 Date: Oct. 10, 2007

(30) Foreign Application Priority Data
Feb. 25, 2005 (KR) 10-2005-0015955

(51) Int. Cl. A61K 39/02; A61K 39/12

(52) U.S. Cl. 424/78.08; 424/200.1; 424/204.1; 514/44

(57) ABSTRACT

The present invention relates to a composition for an immunopotentiator (adjuvant) containing poly-gamma-glutamic acid and a composition for a vaccine containing the immunopotentiator, and more particularly, to an immunopotentiator containing poly-gamma-glutamic acid capable of enhancing antibody production rate by administering it to an animal together with antigen having low immunogenicity, and a composition for a vaccine containing the immunopotentiator and antigen. The inventive adjuvant has almost no toxicity and side effects, and show high antibody titer even when it is used with antigen having poor immunogenicity, so it can be used by adding to medical composition including preventive or curative vaccine for non-contagious chronic diseases as well as cancer, especially prostatic carcinoma, colon carcinoma, lung cancer, breast cancer, ovarian cancer, head and neck cancer, pudendum cancer, bladder cancer, brain tumor and glioma.

### TECHNICAL FIELD

The present invention relates to a composition for an immunopotentiator (adjuvant) containing poly-gamma-glutamic acid and a composition for a vaccine containing the immunopotentiator, and more particularly, relates to an immunopotentiator containing poly-gamma-glutamic acid, which could enhance antibody production rate by administering to an animal with an antigen having low immunogenicity, and a composition for a vaccine containing said immunopotentiator and antigen.

### BACKGROUND ART

Until now, many researches on subunit vaccines using antigenic proteins or peptides, DNA vaccines using antigenic DNA and various recombinant vaccines are being conducted. These vaccine candidate substances have advantage in that they have little side effects, whereas having disadvantage of weak immunogenicity. Thus, the development of immunopotentiator (adjuvant) which efficiently enhances immune reaction of vaccine candidate substances, is urgently needed in this field (O'Hagan, J. Pharm. Pharmacol., 50:1-10, 1998).

Adjuvants are substances capable of enhancing antigen specific humors and/or cell reaction. Humoral reaction (B cell reaction) of an adjuvant shows powerful antibody reaction to specific antigen, and that reaction is known to form deposit which protects antigen from rapid decomposing metabolism and to stimulate an immune reaction non-specifically. When the deposit is formed, the antibody reaction is maintained more persistently, while capacity of an antigen is much smaller by causing long-lasting stimulation to the immune system for a certain amount of time because it could store the antigen and separate it as time passes. And the adjuvant itself stimulates cells of immune system non-specifically so as to have the role of enhancing the reaction with the contained antigen, that is, to have the function of stimulating immune reaction by raising the level of lymphokine.

Because adjuvants have another character of causing powerful T cell mediated immune reaction (cell-mediated response), when administered with an antigen, it is recognized by an antigen presenting cell (APC) to activate immune system, so as to have the use of enhancing the effect of preventive vaccination and remedial vaccination. This adjuvant has the use of non-specific stimulating function having host resistance to infectious diseases and cancer, and enhancing function of immunogenicity of preventive vaccine and remedial vaccine.

Freund's adjuvant is a typical adjuvant among the existing reported adjuvants. Freund's adjuvant is an adjuvant that Arlacel-A, a surfactant is added to mineral, to which a soluble antigen is well mixed to make a suspension and thus injected into a blood vessel or injected hypodermically to enhance the antibody production rate. Freund's adjuvant is the most widely used adjuvant to test animals due to its high antibody production rate, however it has disadvantage in that it can't be used in human medicine because it's highly toxic. In addition, various components showing immunostimulating effect as bacterial products were identified and developed as adjuvants (LPS; lipopolysaccharide, muramyl depeptide, Cholera toxin B subunit), and in the form of QuilA which is a kind of saponin separated from the plant, and immunostimulating complexes (ISCOMs), especially preparations of bile salt and phospholipids etc. were developed as adjuvants. But most of these are preparations whose safety is not ensured.

Currently, aluminum species are almost the only adjuvant approved for use in human patients, but have disadvantage in that immune enhancing effect is relatively low compared to other adjuvants. Also aluminum species mainly enhance humoral immunity by stimulating Th2 immune reaction upon immune reaction (Audibert and Lise, Immunol. Today, 14:281-284, 1993), so it is limited to use as an adjuvant for vaccines requiring the enhancement of cytotoxic T cell immune response. Besides, vaccines containing aluminum adjuvants have disadvantage in that it is difficult to decompose in vivo, and difficult to preserve by lyophilization due to its cohesive, precipitative properties when the aluminum is frozen. In addition, aluminum compounds (aluminum sulfate, aluminum hydroxide, aluminum phosphate etc.) can be used as vaccines for human body but has disadvantage in that the quality is susceptible to change during production, and it is inappropriate for mass production since purifying operation is difficult.

Beside these adjuvants, more safe and effective adjuvants are being developed, and methods, for instance, adjuvants, such as cytokine are administered together with a vaccine antigen are studied. However, these cytokines also need improvement in the safety aspect.

Most of the penetration pathway of virus is through mucosal surface, and many of the infections occur firstly in mucosa and tissue under the mucosa. Since ordinary parenteral vaccines are highly ineffective in inducing mucosal immune response, considerable efforts to develop the system for optimum mucosal immunization have been made. For instance, the development of adjuvants (liposomes, immune stimulating complexes and microsphere) to improve the delivery of an antigen for immunocyte of tissue under the mucosa was attempted (Sjolander et al, J. Leukocyte Biol. 64:713-723, 1998). But even though the mucosal immunization may be effective in many situations, to induce effective immune response in many infections, mucosal and non-mucosal immunizations need to be integrated.

When considering the above arts, to develop commercially viable and available vaccine, an adjuvant, maximizing its effect and able to deliver it safely as well as to mass-produce the selected antigenic substance, must be cost-effective. In addition, an adjuvant capable of transdermal, mucosal and whole body administration, which can control and focus the immune response properly, is needed.

### DISCLOSURE OF THE INVENTION

The present inventors have made extensive efforts to develop a more effective and safe adjuvant, as a result, found that poly-gamma-glutamic acid is useful as an adjuvant by proving that poly-gamma-glutamic acid produced by Bacillus sp., enhances the effect of various antigen and vaccine candidate substances, thereby completing the present invention.

Therefore, the main object of the present invention is to provide a composition for an immunopotentiator (adjuvant) comprising effective dosage of poly-gamma-glutamic acid.

Another object of the present invention is to provide a composition for a vaccine comprising said adjuvant and antigen.

Other features and embodiments of the present invention will be more fully apparent from the following detailed description and appended claims.

### BRIEF DESCRIPTION OF DRAWINGS

FIG. 1 is a graph showing the nucleoprotein antigen-specific IgG antibody titer in serum, a certain amount of time after injecting poly-gamma-glutamic acid and nucleoprotein (N) antigen of porcine transmissible gastroenteritis virus under the skin of rabbits.

FIG. 2 is a graph showing the HBs antigen-specific IgG antibody titer in serum, a certain amount of time after injecting poly-gamma-glutamic acid and surface antigen (L particle) of Hepatitis B virus (HBV) into peritoneal cavity of mice.

FIG. 3 is a graph showing the VP2 antigen-specific IgG antibody titer in serum, a certain amount of time after administering poly-gamma-glutamic acid and lactobacillus surface-expressing capsid antigen protein, VP2 of canine parvovirus, to the mouth and nasal cavity of mice.

FIG. 4 is a graph showing the IgA antibody titer against VP2 antigen in intestinal, and broncho-alveolar lavage fluids of mice, a certain amount of time after administering poly-gamma-glutamic acid and lactobacillus surface expressing VP2 which is a capsid antigen protein of canine parvovirus, to the mouth and nasal cavity of mice.

FIG. 5 is a graph showing the nucleoprotein antigen-specific IgG antibody titer in serum, a certain amount of time after administering poly-gamma-glutamic acid and lactobacillus surface expressing nucleoprotein (N) antigen of porcine transmissible gastroenteritis virus to the mouth of pig together with the feed.

### DETAILED DESCRIPTION OF THE INVENTION

The present invention provides a composition for immunopotentiator (adjuvant) comprising effective dosage of poly-gamma-glutamic acid and pharmaceutically acceptable carrier. In the present invention, it is preferable that molecular weight of said poly-gamma-glutamic acid is 10 kDa˜10,000 kDa.

The present invention also provides a composition for a vaccine comprising the composition for said an immunopotentiator and antigenic substance. In the present invention, said antigenic substance is preferably any one substance selected from the group consisting of peptide, polypeptide, lactobacillus expressing the polypeptide, protein, lactobacillus expressing the protein, oligonucleotide, polynucleotide, recombinant bacteria and recombinant virus. In addition, said antigenic substance is preferably nucleoprotein (N) of porcine transmissible gastroenteritis virus, antigen protein VP2 of canine parvovirus or Hepatitis B surface antigen (L particle), and said nucleoprotein (N) antigenic substance is lactic acid-producing microorganism expressing nucleoprotein (N) and said VP2 antigenic substance is lactic acid-producing microorganism expressing VP2.

The composition for a vaccine according to the present invention additionally comprises at least one second supplement selected from the group consisting of stabilizer, emulsifier, aluminium hydroxide, aluminium phosphate, pH adjuster, surfactant, liposome, iscom supplement, synthetic glycopeptide, extender, carboxypolymethylene, bacterial cell wall, derivatives of bacterial cell wall, bacterial vaccine, animal poxvirus protein, subviral particle supplement, cholera toxin, N,N-dioctadecyl-N′,N′-bis(2-hydroxyethyl)-propanediamin, monophosphoryl lipid A, dimethyl dioctadecyl-ammonium bromide and mixtures thereof. Also, the composition for a vaccine according to the present invention is preferably for the prevention or treatment of at least one disease selected from the group consisting of prostatic carcinoma, colon carcinoma, lung cancer, breast cancer, ovarian cancer, head and neck cancer, pudendum cancer, bladder cancer, brain tumor and glioma.

The present invention also provides a method for enhancing antibody production rate against antigen by administering said composition for a vaccine to animals excluding humans. In the present invention, said animals is preferably mammalia or birds, and administration is preferably performed by any one of the methods selected from the group consisting of hypodermic injection, intramuscular injection, subcutaneous injection, intraperitoneal injection, nasal administration, transdermal administration and oral administration.

An immunopotentiator (adjuvant) comprising poly-gamma-glutamic acid of the present invention may additionally comprise appropriate additives and diluents used generally in the production of pharmacological compositions. Also, an immunopotentiator comprising poly-gamma-glutamic acid according to the present invention may be used by formulating in the form of oral formulations and sterilizing injection solution, such as powders, granules, tablets, capsules, suspension, emulsion, syrup, aerosol etc., respectively by general methods.

Lactose, dextrose, sucrose, sorbitol, mannitol, xylitol, maltitol, starch, glycerin, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate and minerals are carriers, additives, diluents which could be included in an adjuvant composition comprising poly-gamma-glutamic acid.

In case of medical preparations, it could be prepared using commonly used diluents or additives, such as filler, extender, binder, wetting agent, disintegrants, surfactant etc. Solid preparations for oral administration include powders, pills, tablets, granules, capsules etc., and these solid preparations could be prepared by mixing at least one of additives, for example, starch, calcium carbonate, sucrose or lactose, gelatin etc. with said poly-gamma-glutamic acid. Also, besides simple additives, lubricants, such as magnesium stearate talc could be used. Suspension, internal medicine, emulsion, syrup etc. could be used as liquid preparations for oral administration, and in addition to water, liquid paraffin which are commonly used as simple diluents, various additives, for instance, wetting agent, sweetener aromatic agents, preservatives etc. could be included. Preparations for parenteral administration include sterilized solution, non-soluble agent, suspension, emulsion, freeze drying agent. Vegitable oil, such as propylene glycol, polyethylene glycol, olive oil and injectable esters, such as ethyloleate etc. can be used as non-soluble agent and suspension.

In the adjuvant comprising poly-gamma-glutamic acid of the present invention, administration dose could vary according to subject's age, sex, weight etc., and the dose of vaccine administered could vary according to the administration route, severity of diseases, sex, weight, age etc.

The poly-gamma-glutamic acid used in the present invention itself is an adjuvant which can be used safely for prevention because it has almost no toxicity and side effects. The antigenic substances which could be prepared using poly-gamma-glutamic acid of the present invention as an adjuvant for a vaccine, could be selected from the group consisting of antigens with poor immunogenicity or peptides, polypeptides, proteins, or their corresponding DNA sequences, or object cells which is the object of vaccine or a mixture thereof, and could be selected from recombinant bacteria or virus usable as a vaccine.

An immunopotentiator (adjuvant) for a vaccine of the present invention can be used together when administrating a vaccine by parenteral, mucosal (oral and nasal etc.) and transdermal pathways. When using a microorganism expressing antigenic protein as a vaccine, it is preferable to use poly-gamma-glutamic acid of the present invention as an immunopotentiator (adjuvant). Particularly, when using lactobacillus expressing said antigenic protein as an oral vaccine, it is preferable to use poly-gamma-glutamic acid of the present invention together as an immunopotentiator (adjuvant).

Also, poly-gamma-glutamic acid of the present invention can be used by adding it to medical composition comprising preventive or curative vaccine used for preventing and curing non-contagious chronic diseases as well as cancer, especially prostatic carcinoma, colon carcinoma, lung cancer, breast cancer, ovarian cancer, head and neck cancer, pudendum cancer, bladder cancer, brain tumor and glioma.

### EXAMPLES

Hereinafter, the present invention will be described in more detail by specific examples. However, the present invention is not limited to these examples, and it is obvious to those skilled in the art that numerous variations or modifications could be made within the spirit and scope of the present invention.

### Example 1

### Construction of Poly-Gamma-Glutamic Acid

A 5L fermenter containing 3L minimal medium for producing poly-gamma-glutamic acid (GS medium containing 5% L-glutamate, 5% glucose, 1% (NH₄)₂SO₄, 0.27% KH₂PO₄, 0.42% Na₂HPO₄.12H₂O, 0.05% NaCl, 0.3% MgSO₄.7H₂O, 1 ml/L vitamin solution, pH 6.8) was inoculated with 1% of culture broth of Bacillus subtilis var. chungkookjang (KCTC 0697BP), and cultured at a stirring speed of 150 rpm, an aeration rate of 1 vvm, and 37° C. for 72 hours, and then adjusted to pH 3.0 by the addition of 2N sulfuric acid solution, thereby obtaining a poly-gamma-glutamic acid-containing sample solution.

The sample solution was left to stand at 4° C. for 10 hours to remove polysaccharides present in the fermented solution, and added with ethanol to a volume of two times larger than the fermented solution, and then mixed thoroughly. The mixed solution was left to stand at 4° C. for 10 hours, followed by centrifugation, to give poly-gamma-glutamic acid precipitate. The precipitate was dissolved by the addition of distilled water, added with 100 μg/ml protease, and allowed to react in a 37° C. incubator for 6 hours, thereby decomposing extracellular protein present in the sample. The poly-gamma-glutamic acid-containing sample solution was dialyzed against a sufficient amount of distilled water to remove free glutamate, followed by concentration to give pure poly-gamma-glutamic acid. In case of need according to its use, it could be used after producing in a certain molecular weight by cutting said produced poly-gamma-glutamic acid with an appropriate way, or could be used by recovering according to a given molecular weight by an appropriate separating way, and in the following examples 5 kDa, 10 kDa, 20 kDa, 50 kDa, 1000 kDa and 2000 kDa of poly-gamma-glutamic acid was used.

### Example 2

### Production of Antibody Against TGE Virus Antigen by Poly-Gamma-Glutamic Acid

In the present example, to examine if inventive poly-gamma-glutamic acid shows an immune enhancing effect specific to soluble antigen, among the immune responses specific to antibody, especially the effect on humoral immune response by B cell involved in antibody production was examined. Nucleoprotein (N) of Transmissible Gastroenteritis virus (TGE), which induces transmissible digestive organ diseases of pig, was used as an antigen, and rabbits were used as test animals.

Rabbits hypodermically injected with only TGEN antigen (400 μg/PBS ml) were used as a control group, and rabbits hypodermically injected after mixing TGEN antigen (400 μg/PBS ml) and poly-gamma-glutamic acids with molecular weights of 5 kDa, 10 kDa, 20 kDa and 50 kDa, respectively were used as a test group.

Two weeks after the first hypodermic injection, the same amount of antigen and poly-gamma-glutamic acid of each molecular weight was administrated. After the first hypodermic injection, rabbit serum was extracted every 2 weeks, and measured for the titer of antibody against TGEN antigen in serum by ELISA (Enzyme linked immunosorbent assay).

In the ELISA method, serums of the rabbits in the control group and rabbits in the test group were incubated in various series of dilution rate, after blocking TGEN antigen (0.1 μg/ml) coated plate using PBS/5% fetal bovine serum. After that, horse raddish peroxidase conjugated rabbit anti-IgG antibody (specific to Fc) was added. All of the incubation was performed at 37° C. for 1 hour, and after the mentioned each steps, the serums were washed 3 times with PBS/0.05% Tween 20. 30 minutes after developing the reaction by adding ABTS(2,2-azinobis(3-ethylben-zthiazolinesulfonic acid)) 1 mg/ml as a substrate, absorbance at 450 nm was measured with ELISA reader.

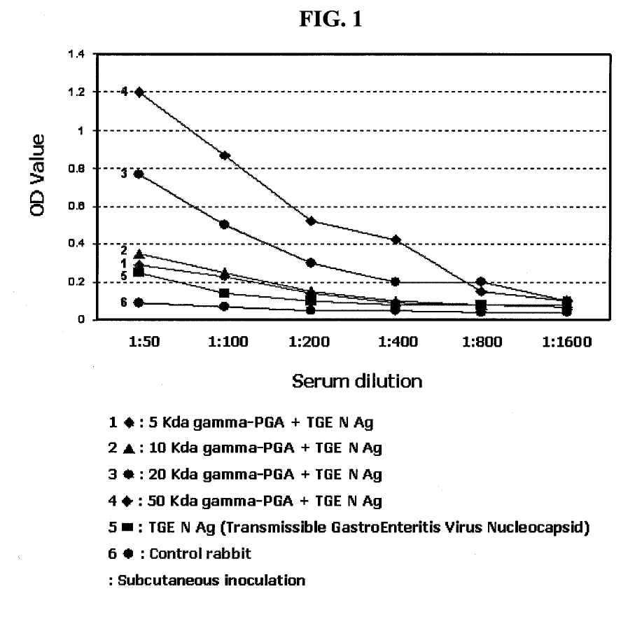

As a result, shown in FIG. 1, in case of administrating poly-gamma-glutamic acids of each molecular weight and TGEN antigen together by hypodermic injection, the antibody titer against TGEN antigen in rabbits was higher compared to that administered only with TGEN antigen by hypodermic injection. Particularly, the antibody titer showed the highest when treated together with 50 kDa poly-gamma-glutamic acid. And the increase in antibody titer was shown to be improved significantly till at least 6 weeks compared to the control group after the first injection.

### Example 3

### Production of Antibody Against HBV Virus Antigen by Poly-Gamma-Glutamic Acid

In the present example, to examine if poly-gamma-glutamic acid shows a specific immune enhancing effect (humoral immune response) on other soluble antigen by intraperitoneal injection, a surface antigen (L particle) of Hepatitis B virus (HBV) derived from yeast were subjected to an experiment using Balb/c mice as a test animal.

As a control group, 6 week-old Balb/c female mice abdominally injected solely with refined HBsAg (hepatitis B virus surface antigen) L particle antigen (1 μg/PBS ml) were used, and for a test group, HBsAg L particle antigen (1 μg/PBS ml) and poly-gamma-glutamic acids(γ-PGA) having molecular weights of 10 kDa, 50 kDa and 1000 kDa, respectively were mixed and abdominally injected. Also, with variations in concentration of antigen, a control group where mice were abdominally injected solely with refined HBsAg L particle antigen (0.5 μg/PBS ml), and a test group injected intraperitoneally with the mixtures of HBsAg L particle antigen (0.5 μg/PBS ml) and poly-gamma-glutamic acids(γ-PGA) having molecular weights of 10 kDa, 50 kDa and 1000 kDa, respectively, were used for the experiment. At 5 weeks after abdominal injection, blood was withdrawn from the test group and control group and HBsAg L particle seroconversion rate in serum and the antibody titer was measured by ELISA (Enzyme linked immunosorbent assay). ELISA was performed the same as the example 2 using a plate coated with HBsAg L particle antigen (1 mg/ml).

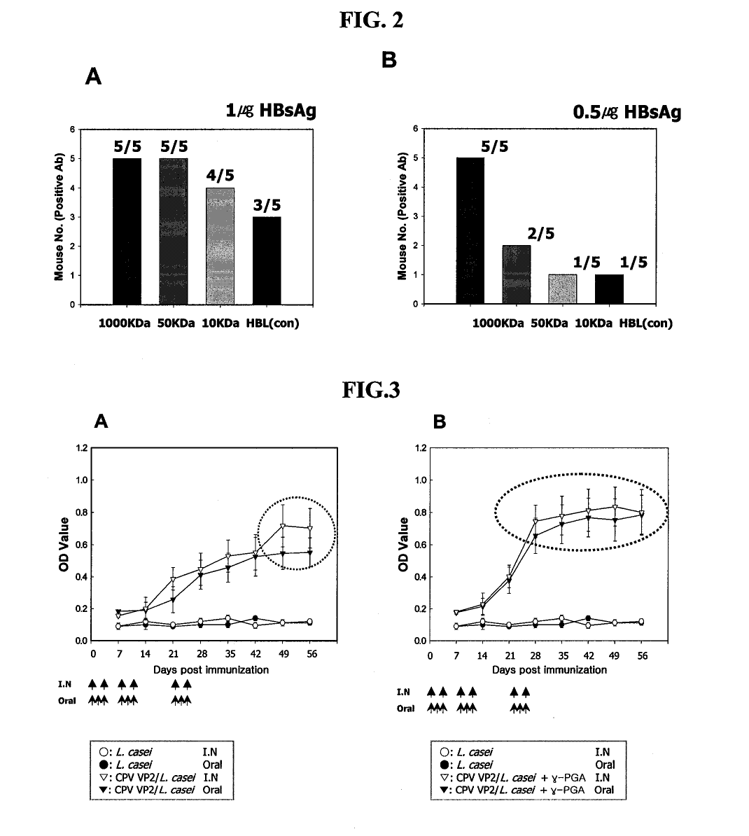

As a result, as shown in FIG. 2, in case of administrating inventive poly-gamma-glutamic acids of each molecular weight together with HBsAg L particle antigen by abdominal injection, anti-HBsAg L particle seroconversion rate of antibody against HBsAg L particle antigen and titer in mice was proportional to the amount of HBsAg L particle antigen, and higher than those of the case hypodermically injected only with antigen. Particularly, seroconversion rate of antibody and titer showed highest when treated together with 1000 kDa poly-gamma-glutamic acid.

### Example 4

### Analysis of Vaccine Effect of lactobacillus Having Canine Parvovirus Antigen Protein Expressed on Surface by Poly-Gamma-Glutamic Acid

In the present example, when a microorganism expressing antigenic protein besides soluble antigens is used as a vaccine, it was examined if the inventive poly-gamma-glutamic acid used as an adjuvant shows an immune enhancing effect specific to antigens (humoral immune response and mucosal immune response).

The capsid antigen protein VP2 of canine parvovirus was used as an antigen. The present inventors have developed lactobacillus having said capsid antigen protein expressed on surface to be used as a new oral vaccine (Korea Patent Application No. 2004-007321). In the present example, antibody production rate of poly-gamma-glutamic acid was examined using lactobacillus having said capsid antigen protein VP2 of canine parvovirus expressed on surface.

Specifically, in the present invention, lactobacillus having capsid antigen protein VP2 of canine parvovirus expressed on surface was collected to a given bacterial concentration, and after the cells were washed with PBS buffer (pH 7.4), lactobacillus, 5×10⁹ cells having the antigen expressed on their surface were orally administered to 4-6-week old C57BL/6 mice five times at an interval of one day, after one week, five times at an interval of one-day, after 2 weeks, five times at an interval of one-day. Also, the Lactobacillus, 1×10⁹ cells having the antigen expressed on their surface were rhinally administered to mice three times at an interval of one-day, after one week, three times at an interval of one-day, after 2 weeks, three times at an interval of one-day and used as a control group. Furthermore, the same group as said control group was prepared to administer 100 μg of 2000 kDa poly-gamma-glutamic acid mixed with each lactobacillus to the mice of the group, thus measuring antibody production rate of capsid antigen protein VP2 in mice of a group without administering poly-gamma-glutamic acid and a group administered with the mixture of lactobacillus and PGA.

After oral administration and rhinal administration, the mouse sera were collected and measured for IgG antibody titer against the capsid antigen protein in serum, and the mouse intestines were collected and measured for IgA antibody titers against the capsid antigen protein in intestinal lavage fluid and bronchoalveolar lavage fluid at an interval of two-weeks using ELISA.

FIG. 3 shows IgG antibody titer against the capsid antigen protein VP2 antigen of canine parvovirus in mouse serum, A shows the antibody titer of a group administered only with lactobacillus having capsid antigen protein VP2 antigen expressed on surface orally and rhinally, and B shows the antibody titer of a group administered orally and rhinally, after lactobacillus having capsid antigen protein VP2 antigen expressed on surface was mixed with poly-gamma-glutamic acid.

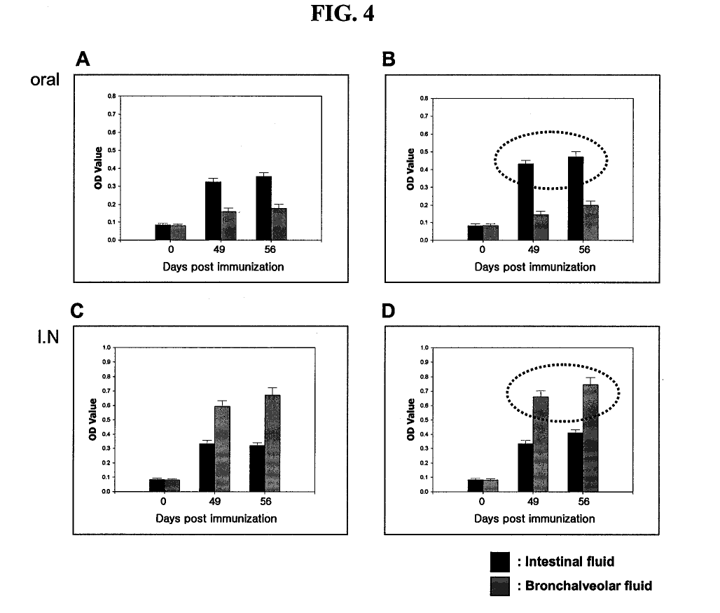

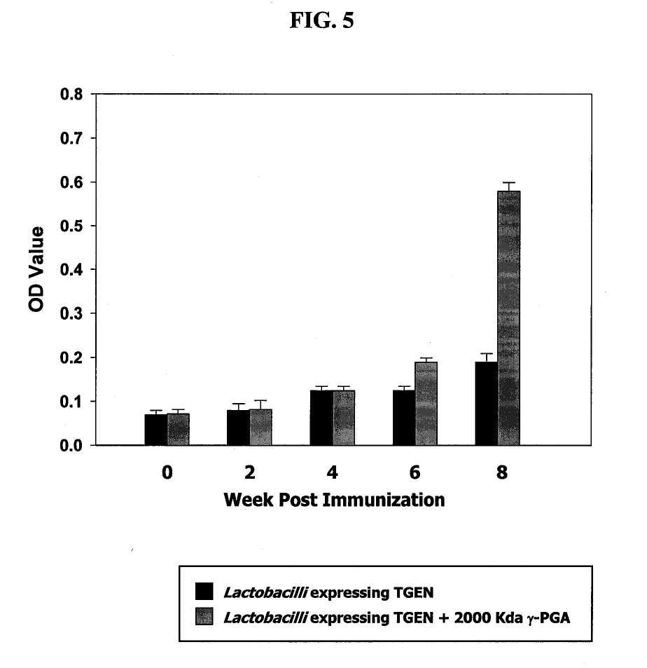

FIG. 4 shows the IgA antibody titer against the capsid antigen protein VP2 antigen in intestinal lavage fluid and bronchoalveolar lavage fluid by ELISA, A and C shows the IgA antibody titer of a group administered only with lactobacillus having capsid antigen protein VP2 antigen expressed on surface orally and rhinally, and B and D shows the IgA antibody titer of a group administered orally and rhinally, after lactobacillus having capsid antigen protein VP2 antigen expressed on surface was mixed with the poly-gamma-glutamic acid.

As shown in FIG. 3 and FIG. 4, when lactobacillus having capsid antigen protein VP2 antigen expressed on surface and the poly-gamma-glutamic acid were administered together, it was confirmed that in the serum, intestinal lavage fluid and bronchioalveolar lavage fluid, the IgG and IgA antibody titers against VP2 antigen which is capsid antigen protein of canine parvovirus were significantly higher than those in the control groups. From these results, it could be found that the poly-gamma-glutamic acid applied in a mixture with the inventive lactobacillus having capsid antigen protein VP2 antigen of canine parvovirus expressed on surface is an adjuvant which could maximize the effect of mucosal vaccines for oral administration.

### Example 5

### Analysis of Vaccine Effect of lactobacillus Having Transmissible Gastroenteritis Virus Antigen Protein Expressed on Surface by Poly-Gamma-Glutamic Acid

In the present example, the effect as an adjuvant was examined when the lacto bacillus having nucleoprotein (N) antigen of transmissible gastroenteritis virus (TGE), which induces transmissible digestive organ diseases of pigs, expressed on surface, was orally administered to pig with poly-gamma-glutamic acid.

Specifically, in the present invention, lactobacillus having nucleocapsid antigen protein N of transmissible gastroenteritis virus expressed on surface was collected to a given bacterial concentration, and after cells were washed with PBS buffer (pH 7.4), lactobacillus having the antigen expressed on their surface were pulverized. The pulverized lactobacillus was mixed with pig's feed at an amount of 0.3% of the pig's feed, and 2 kg/day of the mixed feed was fed to 3 three month old pigs for 4 weeks to use as a control group. 2000 kDa of poly-gamma-glutamic acid was mixed with lactobacillus at an amount of 3% of pulverized lactobacillus, to mix the powder with pig's feed at an amount of 0.3% of pig's feed, and then, 2 kg/day of the mixed feed was fed to 3 three month old pigs for 4 weeks to use as a test group. After the feeding, at an interval of 2 weeks, serum was extracted and measured for IgG antibody titer against the N antigen protein in serum by ELISA.

As a result, as shown in FIG. 5, in the case where a mixture of lactobacillus having nucleocapsid antigen protein N antigen expressed on surface and the poly-gamma-glutamic acid was fed, it could be seen that the IgG antibody titer of serum was high compare to that of the case where nucleocapsid antigen protein N antigen alone was fed. From these results, it was confirmed that the inventive poly-gamma-glutamic acid is an adjuvant which could maximize the effect of mucosal vaccines for oral administration.

Although the present invention has been described in detail with reference to the specific features, it will be apparent to those skilled in the art that this description is solely for a preferred embodiment and does not limit the scope of the present invention. Thus, the substantial scope of the present invention will be defined by the appended claims and equivalents thereof.

### INDUSTRIAL APPLICABILITY

The present invention has an effect of providing a composition for an immunopotentiator (adjuvant) comprising an effective dosage of poly-gamma-glutamic acid. The present invention also has an effect of providing a composition for a vaccine comprising said immunopotentiator and antigen. The inventive adjuvant has almost no toxicity and side effects, and show high antibody titer even when it is used with an antigen having poor immunogenicity, so it can be used by adding it to medical compositions including preventive or curative vaccines for non-contagious chronic diseases as well as cancer, especially prostatic carcinoma, colon carcinoma, lung cancer, breast cancer, ovarian cancer, head and neck cancer, pudendum cancer, bladder cancer, brain tumor and glioma.

1. A composition for an immunopotentiator (adjuvant) comprising an effective dosage of poly-gamma-glutamic acid and a pharmaceutically acceptable carrier.

2. The composition for an immunopotentiator (adjuvant) according to claim 1, wherein the molecular weight of poly-gamma-glutamic acid is in a range of from 10 kDa to 10,000 kDa.

3. A composition for a vaccine comprising the composition for an immunopotentiator according to claim 1, and an antigenic substance.

4. The composition for a vaccine according to claim 3, wherein said antigenic substance is any one substance selected from the group consisting of peptide, polypeptide, lactobacillus expressing the polypeptide, antigen protein, lactobacillus expressing the antigen protein, oligonucleotide, polynucleotide, recombinant bacteria and recombinant virus.

5. The composition for a vaccine according to claim 3, wherein said antigenic substance is nucleoprotein (N) of porcine transmissible gastroenteritis virus, canine parvovirus antigen protein VP2 or surface antigen (L particle) of hepatitis B virus.

6. The composition for a vaccine according to claim 5, wherein said nucleoprotein (N) antigenic substance comprises a lactic acid-producing microorganism expressing nucleoprotein (N) and said VP2 antigenic substance comprises a lactic acid-producing microorganism expressing VP2.

7. The composition for a vaccine according to claim 3, wherein said composition additionally comprises one or more second supplement selected from the group consisting of stabilizer, emulsifier, aluminium hydroxide, aluminium phosphate, pH adjuster, surfactant, liposome, iscom supplement, synthetic glycopeptide, extender, carboxypolymethylene, bacterial cell wall, derivatives of bacterial cell wall, bacterial vaccine, animal poxvirus protein, subviral particle supplement, cholera toxin, N,N-dioctadecyl-N′,N′-bis(2-hydroxyethyl)-propanediamin, monophosphoryl lipid A, dimethyl dioctadecyl-ammonium bromide and mixtures thereof.

8. The composition for a vaccine according to claim 3, for preventing or treating at least one disease selected from the group consisting of prostatic carcinoma, colon carcinoma, lung cancer, breast cancer, ovarian cancer, head and neck cancer, pudendum cancer, bladder cancer, brain tumor and glioma.

9. A method for enhancing antibody production rate against antigen, the method comprises administering the composition of claim 3 to a non-human animal.

10. The method according to claim 9, wherein said animal is selected from among mammalia and birds.

11. The method according to claim 9, wherein said administering comprises a method selected from the group consisting of hypodermic injection, intramuscular injection, subcutaneous injection, intraperitoneal administration, nasal administration, transdermal administration and oral administration.

12. A method for enhancing antibody production rate against antigen in a subject, said method comprising administering to said subject the composition of claim 3.
